(19) United States
(12) Patent Application Publication
(10) Pub. No.: US 2007/0178548 A1
(43) Pub. Date: Aug. 2, 2007

(54) Method for quantitative and semi-quantitative detection of l-phenylalanine, l-tyrosine, l-3,4-dihydroxyphenylalanine and their corresponding keto-acids, phenylpiruvic acid, 3-hydroxyphenylpyruvic acid and 3,4-dihydroxyphenylpyruvic acid in biological fluids, related kits for diagnosis and monitoring of altered metabolisms of these aminoacids

(76) Inventors: Giorgio Ricci, Rome (IT); Francesco Ricci, Rome (IT); Giorgio Federici, Rome (IT)

Correspondence Address: ARENT FOX PLLC, 1050 CONNECTICUT AVENUE, N.W., SUITE 400, WASHINGTON, DC, 20036, US

(21) Appl. No.: 10/580,851

(22) PCT Filed: Nov. 26, 2004
(86) PCT No.: PCT/IT04/00658

§ 371 Date: Dec. 5, 2006

(30) Foreign Application Priority Data
Nov. 28, 2003 (IT) RM2003A000556

(51) Int. Cl. C12Q 1/30

(52) U.S. Cl. 435/27

(57) ABSTRACT

Method for quantitative and semi-quantitative determination of endogenous amino acids L-phenyalanine, L-tyrosine, L-3,4-dihydroxyphenylalanine and their corresponding keto-acids, phenylpiruvic acid, 3-hydroxyphenylpyruvic acid and 3,4-dihydroxyphenylpyruvic in biological fluids useful for diagnosis and monitoring of metabolic disorders of said amino acids or diseases involving said amino acids

The present invention relates to a new method for quantitative and semi-quantitative detection of L-phenylalanine, L-tyrosine, L-3,4-dihydroxyphenylalanine and their corresponding keto acids in biological fluids. A few examples of diagnostic kits are also reported, useful for diagnosis and monitoring of altered metabolisms of these amino acids. More in detail the present invention describes a method for quantitative and semi-quantitative determination of L-phenylalanine (Phe), L-tyrosine (Tyr), L-3,4-dihydroxyphenylalanine (DOPA) and their corresponding keto acids, phenylpiruvic acid (PPA), 3-hydroxyphenylpyruvic acid (HPPA) and 3,4-dihydroxyphenylpyruvic acid (DHPPA), based on a new and specific chemical interaction between these keto acids and a few organic salts of phenazine i.e. phenazine methosulphate (PMS) or phenazine ethosulphate (PES). The reaction products are stable charge transfer complexes that display a peculiar and strong absorption band at about 660 nm allowing the direct quantification of these keto-acids by conventional photometric apparata. The same reaction has been used to quantify the parent amino acids Phe, Tyr and DOPA after their chemical or enzymatic deamination to the corresponding keto acids. The method has been successfully applied for quantifications of these compounds in biological fluids like blood, serum and urine.

It is well known that aminoacids like Phe, Tyr and DOPA are present in biological fluids coming from protein catabolism and also produced ex novo by specific biosynthetic pathways. Their steady-state concentration in biological fluids is tuned by enzymatic and hormonal systems but it may changed as a consequence of congenital or transitory altered metabolisms. Increased levels of these amino acids in blood or serum often yield an increased urinary excretion of the parent keto acids i.e. PPA, HPPA and DHPPA.

Several inborn errors alter the normal concentrations of Phe, Tyr and DOPA and their parent keto acids in biological fluids. They are Phenylketonuria, Hyperphenylalaninemia, Tyrosinosis and Parkinson disease. In addition, it has been reported that during the early phase of neuroblastoma an increased blood concentration of DOPA and an high urinary excretion of DHPPA may also occur.

Phenylketonuria (PKU) is an inherited error of metabolism caused by a deficiency in the enzyme Phe hydroxylase. Loss of this enzyme results in mental retardation, organ damage, unusual posture and can, in cases of maternal PKU, severely compromise pregnancy.

Classical PKU is an autosomal recessive disorder, caused by mutations in both alleles of the gene for Phe hydroxylase (PAH), found on chromosome 12. In the body, Phe hydroxylase converts the amino acid Phe to Tyr, another amino acid. Mutations in both copies of the gene for PAH means that the enzyme is inactive or is less efficient, and the concentration of Phe in the body can build up to toxic levels. In some cases, mutations in PAH will result in a phenotypically mild form of PKU called hyperphenylalaninemia. Both diseases are the result of a variety of mutations in the PAH locus; in those cases where a patient is heterozygous for two mutations of PAH (i.e. each copy of the gene has a different mutation), the milder mutation will predominate.

Classic PKU and the other causes of hyperphenylalaninemia affect about one of every 10,000 births. These disorders are equally frequent in males and females.

Normal blood Phe levels are 58+/−15 micromoles/liter in adults, 60+/−13 micromoles/liter in teenagers, and 62+/−18 micromoles/liter (mean+/−SD) in childhood. In the newborn, the upper limit of normal is 120 micromoles/liter (2 mg/dl) (Scriver et al., 1985; Gregory et al., 1986). In untreated classical PKU, blood levels as high as 2.4 mM/liter can be found.

Newborn screening allows early identification and early implementation of treatment.

The goal of PKU treatment is to maintain the blood level of Phe between 2 and 10 mg/dl. Some Phe is needed for normal growth. This requires a diet that has some Phe but in much lower amounts than normal.

The most commonly used procedure for diagnosis of PKU is the Guthrie test which is a semiquantitative microbiological assay for Phe in blood (Guthrie, R., Susi, A. (1963) Pediatrics 32, 338-343).

The test uses the growth of a strain of bacteria on a specially-prepared agar plate as a sign for the presence of high levels of Phe. The compound B-2-thienylalanine will inhibit the growth of the bacterium Bacillus subtilis (ATCC 6051) on minimal culture media. If Phe is added to the medium, then growth is restored. Such compound will be present in excess in the blood or urine of patients with PKU. If a suitably-prepared sample of blood is applied to the seeded agar plate, the growth of the bacteria in the test will be a positive indicator for PKU in the patient.

To prepare the sample for application, a small amount of blood (from a heel puncture, for example) is applied to a piece of filter paper. Then a small disc is punched from the center of the spot of blood, and the disc applied to the surface of a seeded, minimal-medium agar plate that contains added beta-2-thienylalanine. If the sample contains Phe, then this compound will diffuse into the agar medium. If its concentrations are high enough (as with the excess levels seen with PKU), bacteria will grow under the disc, but not elsewhere. Generally an overnight incubation is enough to determine whether Phe is present in unusual concentrations in blood. Disadvantages of this procedure is the high numbers of false-positive results.

A different analytical method is based on the fluorometric determination of Phe in serum (McCaman, M. N. and Robins, E. (1962) J. Lab. Clin. Med. 9, 885-890) which has been used for newborn screening (Lubenow, N. et al. (1994) Eur. J. Clin. Chem. Clin. Biochem. 32, 525-528). Phe, in the presence of the dipeptide L-leucyl-L-alanine reacts with ninhydrin giving a fluorescent product with an emission maximum at 515 nm (λₑₓ=365 nm). The test may be performed on dried blood samples and analyzed on microplates. The test displays good sensitivity but requires a non-traditional analytical apparatus and it is time expensive.

A different analytical procedure is a spectrophotometric assay based on the oxidative deamination of Phe catalyzed by the enzyme L-phenylalanine dehydrogenase in the presence of NAD⁺. The amount of NADH produced by this reaction is stoichiometric to the amount of Phe. It can be spectrophotometrically quantificated at 500-580 nm on the basis of the reduction of a tetrazolium salt mediated by diaphorase to give a coloured formazan salt. This procedure has been described in 1992 (Dooley, K. C. (1992) Clin. Biochem. 25, 271-276) and subsequently applied to dried spot samples (Randell, E. W. and Lehotay, D. C. (1996) Clin. Biochem. 29, 133-138). Disadvantage of this test is a limit of detection near the cut-off value for Phe which discriminates normal and pathologic samples (0.15 mM). A further problem inherent this procedure is the cross-reactivity of the serum Tyr, which can be used as substrate by L-phenylalanine dehydrogenase. To minimize this interference, the enzymatic deamination step is performed at high pH values, while the reduction of the tetrazolium salt by NADH in the presence of diaphorase is carried out at lower pH values. Thus, a double step procedure is required which complicates the test and is time consuming.

In the last years, a number of chromatographic procedures for the quantification of Phe in biological samples have been also described. Usually they are based on a pre-column amino acid derivatization of Phe with fluorescent reagents like o-pthaldialdehyde. The fluorescent derivative can be separated and quantified by reverse-phase high performance liquid chromatography (Volmer, D. W., Jinks, D. C., Guthrie, R. (1990) Anal. Biochem. 189, 115-121). A capillary zone electrophoresis determination of Phe in serum has been also reported (Tagliaro, F. et al. (1994) Electrophoresis 15, 94-97). All these procedures display good sensitivity but are not useful for routinary screenings because they require non usual instruments and they are expensive and time consuming. Similar disadvantages can be found in other analytical approaches based on mass spectrometry (Sweetman, L. (1996) Clin. Chem. 42, 345-346) and on nuclear magnetic resonance (Novotny, E. J. et al. (1995) Ped. Res. 37, 244-249).

Detection of PPA, which is also present at high concentrations in the urine of the adult PKU patients, is not used for PKU screening. In fact the deamination of Phe, which causes an increased urinary excretion of this keto-acid, can be inefficient in the first one-three weeks from birth. Thus, determination of PPA in urine is useful only for the control of dietary treatments of clear PKU patients. However, sensitive and specific procedures for quantification of urinary PPA have not been described. The simple, but not specific reaction of PPA with ferric chloride in acidic solution has been used in the past for a spot-test on solid support (Phenistix®). This procedure displays low specificity and scarce sensitivity showing a detection limit of 0.6 mM.

Hyperphenylalaninaemia non-PKU can be detected using the Guthrie test or other analytical procedures for Phe determination above described. It is characterized by a moderate increase of serum Phe (<1.2 mM). It is often asynpthomatic, but it has been recently reported that newborns with mothers affected by hyperphenylalaninaemia may have low IQ values and microencephalopathies (Platt, L. D. et al. (2000) Am J Obstet Gynecol 182, 326-33).

Tyrosinaemia type I is a rare genetic metabolic disorder characterized by lack of the enzyme fumarylacetoacetate hydrolase (FAH), which is needed to break down the amino acid Tyr. Failure to properly break down Tyr leads to abnormal accumulation of Tyr and its metabolites in the liver, potentially resulting in severe liver disease. Tyr may also accumulate in the kidneys and central nervous system.

Symptoms and physical findings associated with Tyrosinaemia type I include failure to gain weight and grow at the expected rate (failure to thrive), fever, diarrhea, vomiting, an abnormally enlarged liver (hepatomegaly), and yellowing of the skin and the whites of the eyes (jaundice). Tyrosinaemia type I may progress to more serious complications such as severe liver disease. Tyrosinaemia type I is inherited as an autosomal recessive trait.

Tyrosinaemia type II (oculocutaneous tyrosinaemia) is an autosomal recessive disorder resulting from a deficiency in Tyr aminotransferase. Skin, eye and neurological signs are the cardinal features of this disease. Skin manifestation usually begins after the first year of life, but may begin as early as 1 month of age. Patients generally suffer from progressive, painful, non-pruritic and hyperkeratotic plaques on the soles and palms. Leucokeratosis of the tongue has also been reported.

Both these pathologies, Tyrosinaemia type I and II, can be detected by determining the serum concentration of L-Tyr or the urine concentration of HPPA, the parent keto acid of Tyr.

HPPA has been detected in urine by means of a colorimetric procedure (Millon test) which is not specific and displays scarce sensitivity. More accurate methods use high performance liquid chromatography or the specific reaction of L-Tyr with α-nitroso-β-naftolo. The fluorescent product can be measured at 570 nm (excitation at 460 nm) (Hsia, D., Y.-Y., Inouye, T. in “Inborn Errors of Metabolism” (Yearbook Medical Publisher, Inc) pp 69-71 (1966))

Neuroblastoma is one of the most common solid tumours of early childhood usually found in babies or young children. The disease originates in the adrenal medulla or other sites of sympathetic nervous tissue. The most common site is the abdomen (near the adrenal gland) but can also be found in the chest, neck, pelvis, or other sites. Most patients have widespread disease at diagnosis.

In about 90% of cases of neuroblastoma, elevated levels of catecholamines or its metabolites are found in the urine or blood. Recent investigations also showed that this disease also causes high plasma DOPA concentrations and increased excretion of DOPA and of its catabolites (Ikeda H. et al (1996) Pediatr Hematol Oncol. 13, 21-32; Ikeda, H et al. (1995) Hypertens. Res. 18, S209-210).

Thus, plasma DOPA may be a useful marker in the diagnosis and follow-up of neuroblastoma.

A number of analytical procedures for the determination of DOPA in blood or urine have been described previously. Most of these tests are based on DOPA derivatization with fluorescent reagents, separation by HPLC and fluorometric determination (Turler K, Kaser H. (1971) Clin Chim Acta. 32, 41-51; Liu Y. et al. (2003) Journal of Fluorescence 13, 123-128). Conversely, no simple or sensitive procedures are available for quantification of DHPPA.

Parkinson's disease is a disorder that affects nerve cells (neurons) in the part of the brain controlling muscle movement. People with Parkinson's often experience trembling, muscle rigidity, difficulty walking, and problems with balance and coordination. These symptoms generally develop after age 50, although the disease affects a small percentage of younger people as well.

Parkinson's disease is progressive, meaning the signs and symptoms become worse over time. But although Parkinson's may eventually be disabling, the disease often progresses gradually, and most people have many years of productive living after a diagnosis.

Furthermore, unlike other serious neurologic diseases, Parkinson's disease is treatable. For decades, the drug levodopa, commonly known as L-DOPA, has been the mainstay of Parkinson's disease treatment. But DOPA can cause side effects, and it may become less effective as the disease worsens, especially as new symptoms develop. Efficient procedures for the quantification of DOPA and its metabolites in blood, serum or urine to optimize pharmacologic treatments for this disease have been reported. For example a sensitive method the determination of L-DOPA in plasma and cerebrospinal fluid has been optimized using gas chromatography and electron capture negative ion mass spectrometry. (de Jong A P et al. (1988) Clin Chim Acta. 171, 49-61). Other methods are based on liquid chromatography-tandem mass spectrometry (Igarashi K. et al. (2003) J Chromatogr B Analyt Technol Biomed Life Sci. 792, 55-61), gas-chromatographic determinations (Imai K, et al. (1972) Chem. Pharm. Bull. 20, 2436-2439), liquid-chromatographic assay (Eisenhofer, G. et al. (1986) Clin. Chem. 32, 2030-2033). All these procedures require expensive apparata and are time consuming.

In conclusion, it appears important the discovery and standardization of new analytical procedures that are fast, sensitive, and limit the costs of the analyses for diagnosis and monitoring of dismetabolisms involving Phe, Tyr and DOPA.

The authors of the present invention have discovered that a few phenazine derivatives, i.e. PMS and PES react specifically with PPA, HPPA, and DHPPA giving charge transfer complexes which strongly absorb in the visible region showing a maximum at about 663 nm. The reaction occurs at alkaline pH values (between pH 7.0 and pH 11) and the colored product is stable for hours when protected from light. As both PMS, PES and the above mentioned keto acids do not absorb at this wavelength, this reaction has been successfully employed to standardize new analytical procedures for a simple quantification of these keto acids in biological fluids. Furthermore, the same reaction has been used to quantify Phe, Tyr and DOPA after previous enzymatic deamination of these amino acids to give PPA, HPPA and DHPPA. The authors proved that this specific reaction can be used either in solution and on solid supports.

A few analytical details of this interaction are reported for sake of clarity.

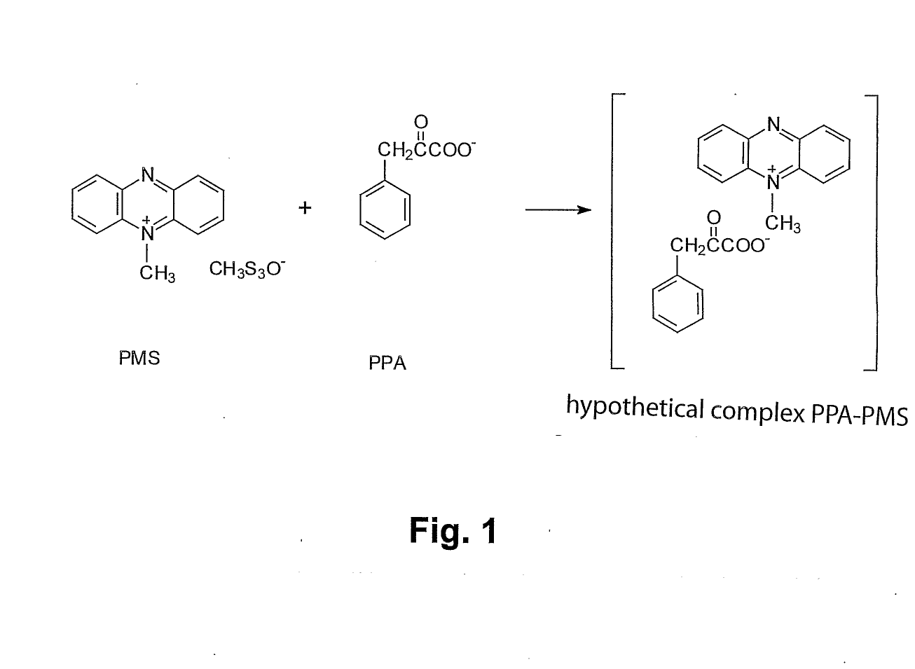

The spectral analysis of the interaction product between PMS and PPA is shown in FIG. 1. After 20 min incubation of 0.1 mM PPA with 1 mM PMS 1 ml of 1 M potassium phosphate buffer, pH 8.5 (25° C.), the absorbance at 663 nm is stable for hours. The blue product is moderately soluble in buffered solution.

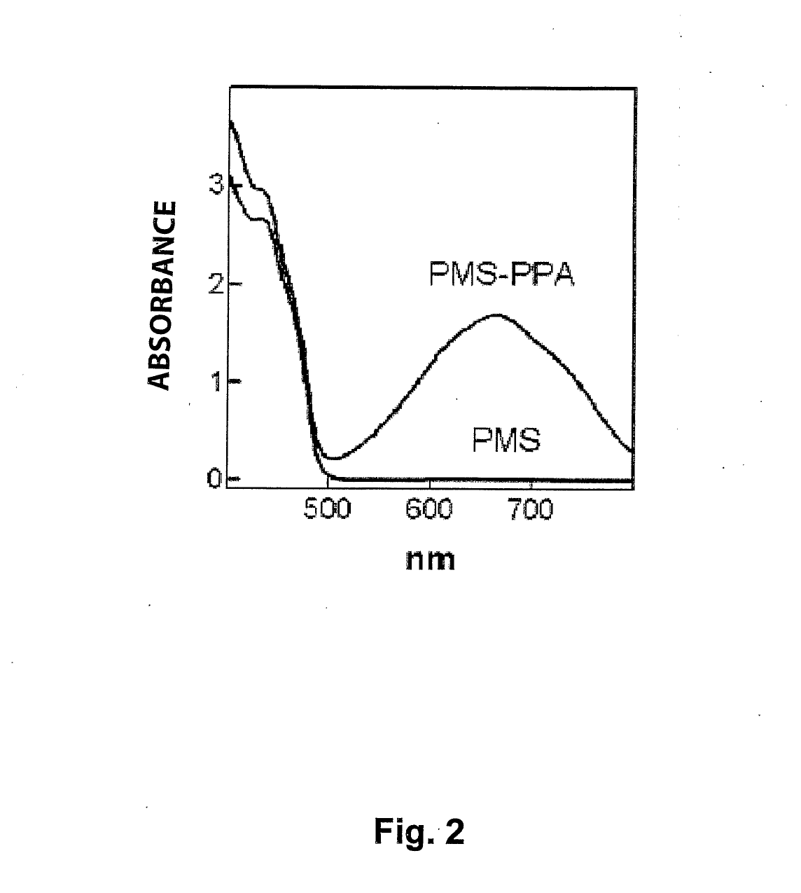

Chemical characterization of the interaction product between PMS and PPA has been performed using mass spectrometry and UV spectral analysis . After reaction of 0.1 M PMS with 0.1 M PPA at pH 8.5, the colored product appears as a deeply blue precipitate. The scarcely soluble product has been crystallized two fold from water. The visible spectrum at pH 8.5, obtained for solubilization of the blue precipitate in potassium phosphate buffer, pH 8.5, overlaps the spectrum reported in FIG. 1 and the mass spectrum gives a molecular ion at 358, suggesting a complex 1:1 between PMS and PPA. A second peak at 397 is also present in the mass spectrum indicating that the complex is partially present as a potassium salt. On the basis of the peculiar absorption band of the interaction product at high wavelength, it can be identified as a charge transfer complex. Possible reaction scheme is shown in FIG. 2.

The authors discovered that very similar reaction occurs by reacting PMS with HPPA e DHPPA in alkaline conditions. The interaction products obtained in 1 M potassium phosphate buffer, pH 8.5, display absorption spectra very similar to the one due to the PMS-PPA complex (not shown). A similar process also occur by replacing PMS with PES (not shown) giving identical visible spectra.

Kinetics of formation of these PMS or PES complexes strictly depends on the ionic strength of the solution. As shown in Table 1, a faster reaction occurs when the concentration of the phosphate buffer is high and the highest velocity has been observed when the concentration of the buffer is 1 M.

TABLE 1

| | Initial rate at 663 nm (□A/min) |
|---|---|
| Buffer concentration (M) | (0.1 M PPA- 1 mM PMS) |
| 1 | 0.19 |
| 0.5 | 0.15 |
| 0.1 | 0.05 |
| 0.05 | 0.03 |
| 0.01 | 0.005 |

The peculiar reaction described in the present invention is highly specific for PPA, HPPA and DHPPA. In fact a lot of different endogeneous keto acids, including pyruvic acid, oxabacetic acid, ketoglutaric acid, and ketobutirric acid did not gave any apparent reaction with PMS or PES.

The high stability and the high value of the extinction coefficients of the charge transfer complexes together with lack of interferences by other endogeneous keto-acids, make this reaction useful for quantitative determinations of PPA, HPPA and DHPPA in biological fluids. In addition this reaction can be used to quantify the parent amino acids Phe, Tyr and DOPA after previous enzymatic or chemical deamination to give PPA, HPPA and DHPPA that are then reacted with PMS or PES.

Furthermore, the use of selected buffers to be included in the reaction mixture allows more accurate quantifications of the single amino acid or keto-acid. In fact, the authors discovered that the extinction coefficients of the charge transfer complexes also depends on the chemical composition of the buffer used in the incubation mixture. As shown in Table 2, the highest extinction coefficients for PMS-PPA, PMS-HPPA and PMS-DHPPA complexes have been found when the reaction is performed in potassium phosphate buffers, while TRIS-HCl buffer causes a dramatic lowering of the extinction coefficients of PMS-HPPA and PMS-DHPPA complexes. This property has been used by the authors to discriminate the amount of PPA from the amount of DHPPA+HPPA in biological samples.

TABLE 2

| Buffer (1 M) | ε PPA | ε HPP | ε DHPP |
|---|---|---|---|
| Phosphate pH 8.5 | 17000 | 13500 | 13500 |
| Phosphate pH 8.0 | 16000 | 12000 | 12000 |
| Phosphate pH 7.5 | 12000 | 10000 | 10000 |
| TRIS-HCl pH 8.5 | 16500 | 3500 | 3500 |
| TRIS-HCl pH 8.0 | 14000 | 2500 | 2500 |
| TRIS-HCl pH 7.5 | 10000 | 1000 | 1000 |
| Glycine-NaOH pH 8.5 | 9000 | 1300 | 1200 |
| Glycine-NaOH pH 9.5 | 7000 | 600 | 600 |
| Borate pH 8.5 | 9000 | 2200 | 2000 |
| Borate pH 9.5 | 8000 | 2700 | 2600 |

Thus, the specific object of the present invention is a new method for quantitative and semi-quantitative determination of endogenous amino acids L-phenyalanine, L-tyrosine, L-3,4-dihydroxyphenylalanine and their corresponding keto-acids, phenylpiruvic acid, 3-hydroxyphenylpyruvic acid and 3,4-dihydroxyphenylpyruvic in biological fluids, such as urine, blood or serum, useful for diagnosis and monitoring of metabolic disorders of said amino acids or diseases involving said amino acids, comprising the following steps:

a) reaction of phenylpiruvic acid, 3-hydroxyphenylpyruvic acid and 3,4-dihydroxyphenylpyruvic, present as such in biological fluids or coming from the parent endogenous amino acids L-phenyalanine, L-tyrosine, L-3,4-dihydroxyphenylalanine by deamination, with an organic salt of phenazine derivatives in the presence of at least one alkaline buffer to give colored charge transfer complexes;

b) measurement of the absorbance values due to said charge transfer complexes in the wavelength range from 650 to 690 nm and quantification of the keto acids or amino acids concentrations in biological fluids.

The phenazine derivatives useful for the present invention are selected from the group consisting of phenazine methosulphate (PMS) or phenazine ethosulphate (PES).

According to a particular embodiment, when the biological fluid is serum, the endogeneous amino acids are previously deaminated by means of a chemical or enzymatic reaction.

The deamination reaction of the endogenous amino acids can be catalyzed by the enzyme L-amino acid oxidase in the presence of an alkaline buffer.

When the endogenous amino acid to be determined is L-phenyalanine, the deamination reaction can be performed using the enzyme L-phenyalanine dehydrogenase in alkaline conditions and in the presence of a redox cofactor, such as for example NAD⁺.

Classical alkaline biological buffers can be used for the reaction (step a) of salt of phenazine derivatives such as PMS or PES, with PPA, HPPA, DHPPA, for example, potassium phosphate buffer, sodium phosphate buffer, TRIS-HCl buffer.

The reaction (step a)) can be performed in solution or on solid support. In a particular application of the present invention quantification of PPA, HPPA, DHPPA or their parent amino acids has been obtained after separation of the corresponding charge transfer complexes, for example by using a reverse-phase column and a high performance liquid chromatographic apparatus provided of a photometric detector set at 663 nm.

The method according to the present invention may be usefully employed for diagnosis or control of many metabolic disorders or diseases involving L-phenyalanine, L-tyrosine, L-3,4-dihydroxyphenylalanine such as, for example, Phenylketonuria, Hyperphenylalaninaemia, Tyrosinaemia, Neuroblastoma, Parkinson disease.

Thus, specific object of the present invention is also a kit for diagnosis and monitoring of metabolic disorders of L-phenyalanine, L-tyrosine, L-3,4-dihydroxyphenylalanine or diseases involving said amino acids, based on quantitative detection of the parent keto-acids phenylpiruvic acid, 3-hydroxyphenylpyruvic acid and 3,4-dihydroxyphenylpyruvic comprising:

a) an organic salt of phenazine derivative in solution or adsorbed on solid support such as, for example, cellulose or equivalent absorbing materials;

b) an alkaline buffer such as the extinction coefficients at 663 nm of complexes of phenylpiruvic acid, 3-hydroxyphenylpyruvic acid and 3,4-dihydroxyphenylpyruvic with an organic salt of phenazine derivatives are higher than 13,000 M⁻¹ cm⁻¹. The organic salt of phenazine derivative can be phenazine methosulphate (PMS) or phenazine ethosulphate (PES). The alkaline buffer b) can be potassium phosphate buffer or sodium phosphate buffer.

According to a particular embodiment of the present invention, the kit comprises also:

c) a second alkaline buffer which lowers the extinction coefficients at 663 nm of complexes of phenylpiruvic acid, 3-hydroxyphenylpyruvic acid and 3,4-dihydroxyphenylpyruvic with an organic salt of phenazine derivatives below 3,600 M⁻¹ cm⁻¹. The second alkaline buffer c) can be selected from the group consisting of TRIS-HCl buffer, glycine-NaOH buffer, borate buffer.

The diagnostic kits can be used for the quantification of Phe, Tyr or DOPA in biological fluids after enzymatic or chemical deamination step to give the corresponding keto acids (PPA, HPPA and DHPPA) and subsequent reaction with PMS or PES. In this particular application, the kit includes a deaminating enzyme like L-aminoacid oxidase. In a particular application for the quantification of Phe alone, the kit includes a more specific deaminating enzyme, i.e. L-phenylalanine dehydrogenase, in the presence of a redox coenzyme like NAD⁺.

Various aspects of the present invention will be further illustrated by the following non-limiting examples and by the included figures, wherein:

FIG. 1 shows the reaction scheme of PMS with PPA to give the charge transfer complex PMS-PPA.

FIG. 2 shows the spectrum of complex PMS-PPA after 20 min of incubation of 0.1 mM PPA with 1 mM PMS in 1 M potassium phosphate buffer, pH 8.5. The spectrum of 1 mM PMS in the same buffer is also shown. The maximum of absorbance of the PMS-PPA complex is at 663 nm.

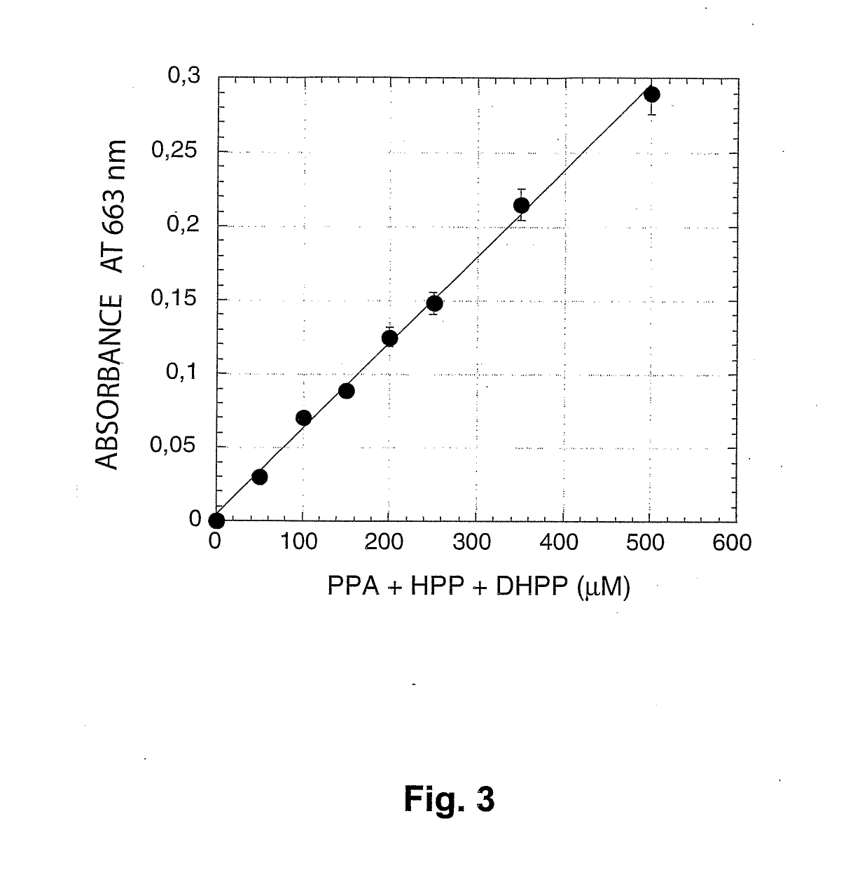

FIG. 3 shows the dependence of the absorbance at 663 after reaction of variable amounts of PPA, HPPA and DHPPA with 1 mM PMS in the same conditions reported in the Example 1. Absorbance values (arbitrary units) come from reflectance measurements at 663 nm.

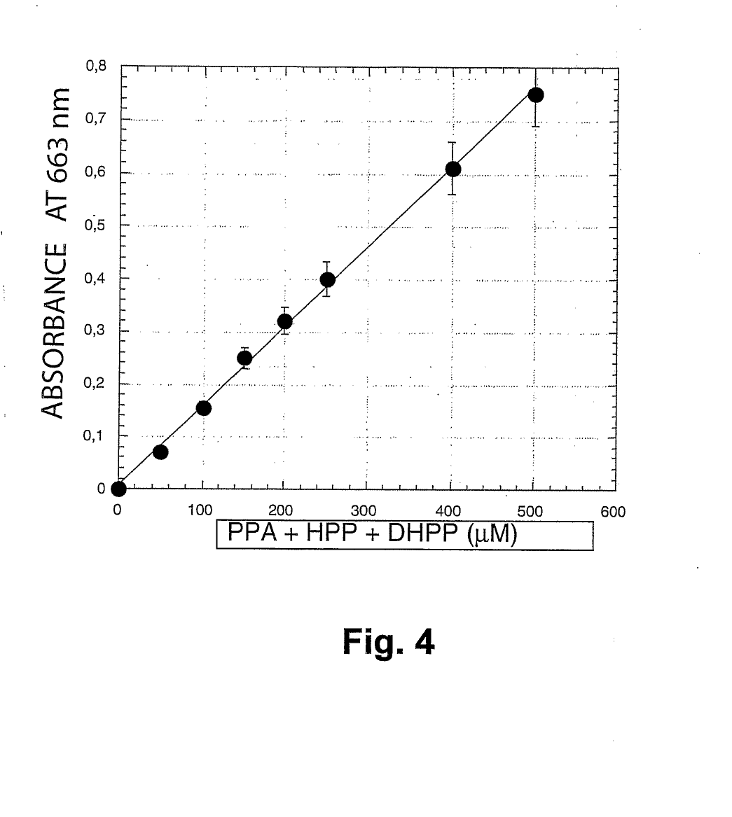

FIG. 4 shows the absorbance values at 663 nm after incubation of variable amounts of PPA+HPPA+DHPPA in urine with 1 mM PMS in 1 M potassium phosphate buffer, pH 8.5, according to Example 3.

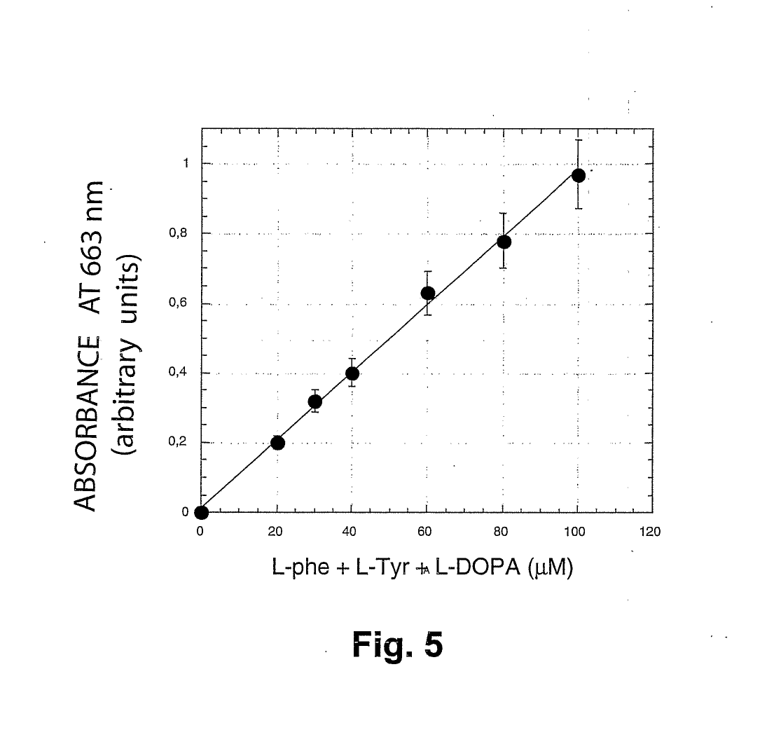

FIG. 5 shows the absorbance values at 663 nm after enzymatic deamination of different amounts of Phe+Tyr+DOPA present in serum and subsequent reaction on solid support, according to Example 5. Absorbance values (arbitrary units) came from reflectance measurements at 663 nm.

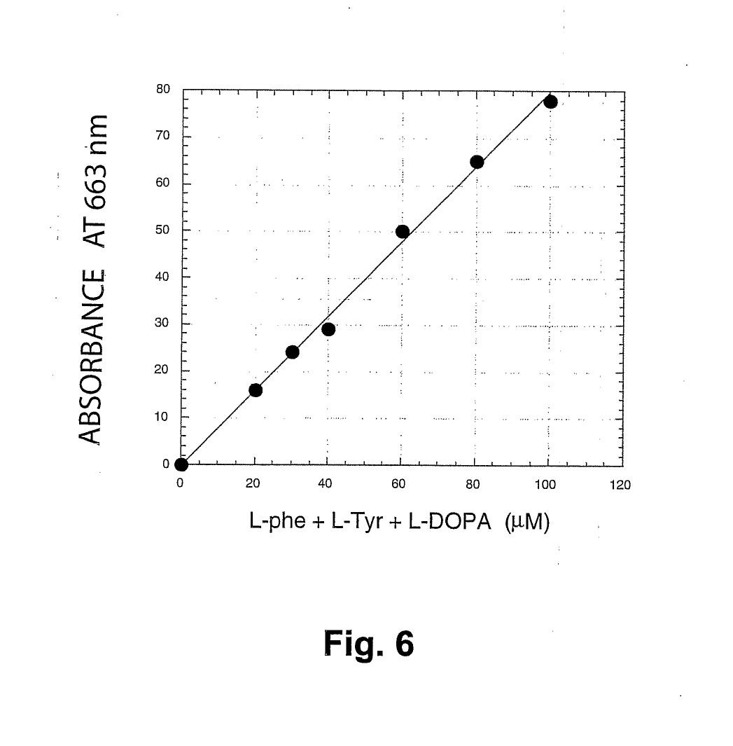

FIG. 6 shows the absorbance at 663 nm (arbitrary units) after enzymatic deamination of different amounts of Phe+Tyr+DOPA and subsequent reaction in solution with 1 mM PMS in 1 M potassium phosphate buffer, pH 8.5, according to Example 6.

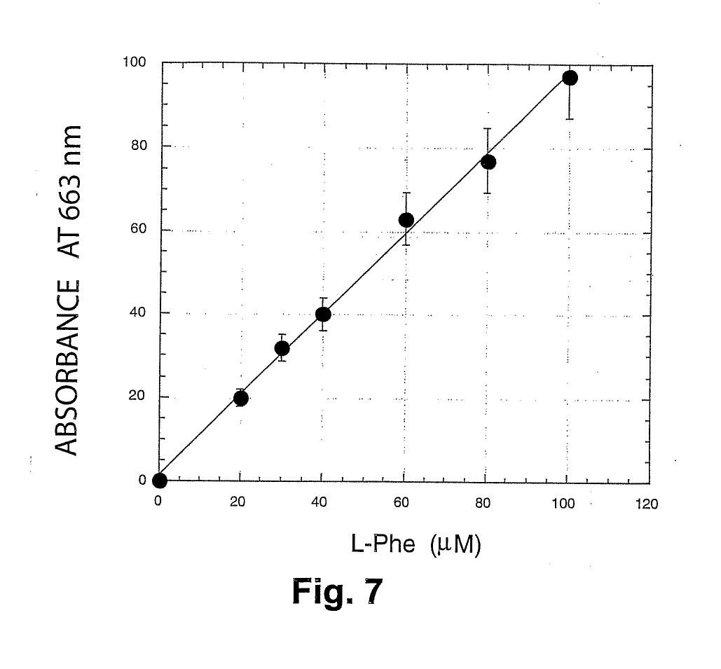

FIG. 7 shows the absorbance at 663 nm (arbitrary units) after enzymatic deamination of variable amounts of serum Phe and subsequent reaction with PMS on solid support, according to Example 7. Data are given by reflectance measurements

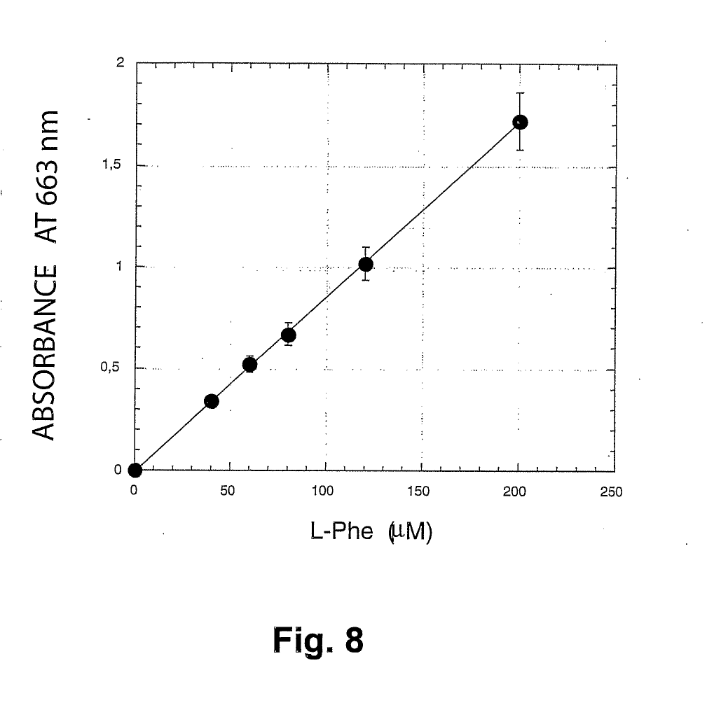

FIG. 8 shows the absorbance at 663 nm (arbitrary units) after enzymatic deamination of variable amounts of serum Phe and subsequent reaction with 1 mM PMS in solution, according to Example 8.

### EXAMPLE 1

### Test for Semi-Quantitative Determination of Urinary PPA+HPPA+DHPPA on Solid Support

a) Preparation of the Diagnostic Stick:

Cellulose discs (2 cm diameter) were plunged into an ethanol solution containing 10 mM PMS. After 2 min, the discs were dried in the dark under air stream (30° C.). The diagnostic stick is stable for at least one year when stored in the dark.

b) Test Procedure:

1 ml of urinary sample is diluted with 1 ml of 1 M potassium phosphate buffer, pH 8.5. 0.1 ml of this urinary sample is laid on the diagnostic stick prepared as described in a). The presence of PPA or HPPA or DHPPA is signaled by the appearance of a blue spot which gives a stable color within 20 min. Comparison of color scale gives a semi-quantitative estimation of the urinary concentration PPA+HPPA+DHPPA. The limit of detection is 30 μM of keto acids. A more accurate quantitative estimation can be obtained by reflectance measurements at 663 nm. FIG. 3 shows spectral data obtained using urinary samples implemented with variable amounts of PP, HPPA and DHPPA. The observed reflectance data were converted into absorbance values and subtracted by the blank values. Experimental points represent the mean of four different measurements and standard errors are reported

### EXAMPLE 2

### Test for Quantitative Determination of Urinary PPA and HPPA+DHPPA on Solid Support

1 ml of urinary sample is diluted with 1 ml of 1 M TRIS-HCl buffer pH 8.5 and a 0.1 ml aliquot is laid on the diagnostic stick containing PMS, prepared as described in Example 1. The same urine sample is also diluted in 1 M potassium phosphate buffer and analyzed as described in Example 1. As the extinction coefficients of PMS-HPPA and PMS-DHPPA complexes are dramatically lowered in the presence of TRIS-HCl buffer (ε=3,500 M⁻¹ cm⁻¹, a comparison of the absorbance values at 663 (caming from reflectance measurements) makes possible the quantification of PP and HPP+DHPP on the basis of a simple mathematical system composed by two equations and two unknowns. Table 3 shows some optical data obtained with urine samples implemented of variable amounts of PPA and HPPA+DHPPA

TABLE 3

| | | Absorbance | Absorbance | | |
|---|---|---|---|---|---|
| | | (Test-BK) | (Test-BK) | PPA | HPP + DHP |
| PP | HPP + DHPP | (arbitrary units) | (arbitrary units) | Calculated | Calculated |
| (μM) | (μM) | Phosphate buffer 1M | TRIS-HCl buffer 1M | (μM) | (μM) |
| 100 | 0 | 1.70 ± 0.05 | 1.60 ± 0.05 | 92 | 8 |
| 0 | 100 | 1.35 ± 0.05 | 0.30 ± 0.05 | 0 | 100 |
| 50 | 50 | 1.52 ± 0.05 | 1.05 ± 0.05 | 53 | 47 |
| 100 | 50 | 2.37 ± 0.04 | 1.87 ± 0.05 | 98 | 51 |
| 150 | 50 | 3.31 ± 0.05 | 2.72 ± 0.07 | 148 | 59 |
| 50 | 100 | 2.25 ± 0.03 | 1.20 ± 0.06 | 47 | 105 |
| 100 | 100 | 3.09 ± 0.06 | 2.10 ± 0.09 | 107 | 96 |

### EXAMPLE 3

### Test for Semi-Quantitative Determination of Urinary PPA+HPPA+DHPPA in Solution

0.1 ml of urine sample is added to 0.9 ml of 1 mM PMS dissolved in 1M potassium phosphate buffer, pH 8.5, containing 1 mM EDTA. After 20 min, the solution is analyzed spectrophotometrically at 663 nm. The absorbance values were subtracted by the absorbance values observed by replacing the urinary samples with 0.1 ml of deionized water (BK). By assuming an extinction coefficient similar for PMS-PPA, PMS-HPPA and PMS-DHPPA complexes (15,000 M⁻¹ cm⁻¹), an approximate estimation of the urine concentration of PP+HPPA+PMS-DHPPA can be obtained. FIG. 4 shows the linear dependence of the absorbance at 663 nm on increasing urine concentrations of PPA, HPPA and DHPPA. The detection limit of this procedure is 3 μM of PP+HPPA+DHPPA, which correspond to a urinary concentration of 30 μM.

### EXAMPLE 4

### Test for Quantitative Determination of Urinary PPA and HPPA+DHPPA in Solution

The procedure is based on a comparison of the absorbance values at 663 nm obtained according to the procedure of the Example 3 and the absorbance values at 663 nm observed when the urine samples are reacted with PMS in 1 M TRIS.-HCl buffer, pH 8.5. The extinction coefficients at 663 nm of the PMS-HPPA and PMS-DHPPA complexes are lowered below 3,600 M⁻¹ cm⁻¹ in TRIS-HCl buffer. Thus, an approximate estimation of PP and HPPA+DHPPA in the urine sample can be obtained on the basis of a simple mathematical system composed by two equations and two unknowns. The test may be realized either as a kinetic or end-point assay. In addition, the test has been optimized by using both a traditional spectophotometric apparatus, or an automated microplate spectrophotometric ELISA apparatus with a filter at 660 nm. In the last case, the final volume of the test solution is 0.3 ml.

### EXAMPLE 5

### Test for Semi-Quantitative Detection of Serum Phe+Tyr+DOPA on Solid Support

The total concentration of Phe+Tyr+DOPA in serum has been evaluated after enzymatic deamination of these aminoacids by L-amino acid oxidase (L-AAO) (EC 1,4,3,2) and subsequent reaction of the parent keto acids with PMS on solid support.

The enzymatic reaction can be illustrated as follows: 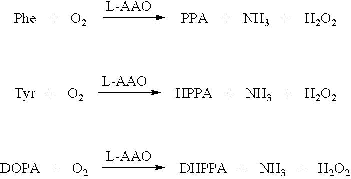
PPA, HPPA and DHPPA are quantified by following the same procedure reported in Example 1.

0.5 ml of serum samples, containing variable and equimolar amounts of Phe, Tyr and DOPA, were diluted with 0.5 ml of 1 M potassium phosphate buffer, pH 8.5 containing 0.2 units of commercial L-amino acid oxidase (Sigma, USA). After 30 min incubation at 20° C., 0.1 ml aliquots were loaded on the cellulose stick containing PMS, according to the procedure reported in the Example 1. When the color development is complete (within 20 min), the stick can be analyzed by a reflectance apparatus set at 663 nm. The observed reflectance data were converted into absorbance values and subtracted by the blank values. Data are reported in FIG. 5.

### EXAMPLE 6

### Test for Semi-Quantitative Determination of Serum Phe+Tyr+DOPA in solution

0.5 ml of serum samples, containing variable end equimolar amounts of Phe, Tyr and DOPA, were diluted with 0.5 ml of 1 M potassium phosphate buffer, pH 8.5 containing 0.2 units of commercial L-amino acid oxidase (Sigma, USA) and 1 mM PMS. After 30 min incubation at 20° C. each sample was analyzed spectrophotometrically at 663. The observed absorbance values at 663 nm were subtracted by the absorbance values observed in identical experiments without L-amino acid oxidase. The concentration of Phe+Tyr+DOPA was estimated by assuming an average extinction coefficient of 15,000 M⁻¹ cm⁻¹ at 663 nm for all the parent keto acids in complex with PMS. FIG. 6 shows the dependence of the observed absorbances at 663 nm on the total concentration of Phe+Tyr+DOPA.

### EXAMPLE 7

### Test for Quantitative Analysis of Serum Phe on Solid Support

Quantification of serum Phe has been obtained by using a procedure similar to the one reported in Example 5, replacing L-amino acid oxidase with suitable amounts of the more specific enzyme, L-Phe dehydrogenase and suitable amounts of NAD⁺ as coenzyme. 0.5 ml of human serum samples, containing variable amounts of Phe were diluted with 0.5 ml of 1 M potassium phosphate buffer, pH 8.5 containing 1 unit of commercial L-phenylalanine dehydrogenase (Sigma, USA) and 0.4 mM NAD⁺. After 30 min incubation at 37° C., 0.1 ml aliquots were loaded on the cellulose stick containing PMS, according to the procedure reported in the Example 1. The color development is complete within 20 min and the stick can be analyzed by using a reflectance apparatus set at 663 nm. The reflectance data were converted into absorbance values and subtracted by the blank values. Data are shown in FIG. 7. Under these specific conditions, the cross-reactivity of Tyr and DOPA does not exceeds 1%.

### EXAMPLE 8

### Test for Quantitative Analysis of Serum Phe in Solution

0.5 ml of human serum samples, containing variable amounts of Phe were diluted with 0.5 ml of 1 M potassium phosphate buffer, pH 8.5 containing 1 unit of commercial L-Phe dehydrogenase (Sigma, USA), 0.4 mM NAD⁺ and 2 mM PMS. After 30 min incubation at 20° C., the samples were analyzed spectrophotometrically at 663 nm. Absorbance values were subtracted by the absorbance values observed in identical experiments without L-phenylalanine dehydrogenase. FIG. 8 shows the dependence of the observed absorbance values at 663 nm on serum Phe concentration.

### EXAMPLE 9

### Test for Neonatal Screening of Phenylketonuria

Specimen collection and storage: heel stick blood (normal or implemented with known amounts of Phe) has been collected on Shleicher and Schuell 903 filter paper, or equivalent. The specimens are dried at room temperature away from direct sunlight, dust, moisture and heat. The specimens are stable at least one year when stored at 2-7° C.

From each specimen, 4 mm diameter spots were punched. Each spot was immersed into absolute methanol to fix blood proteins and dried at room temperature. The dried spots were incubated with 0.5 ml of 1 M phosphate buffer, pH 8.5 (or 1 M Tris-HCl buffer, pH 8.5) and shaken for 10 min. 0.19 ml of this solution were placed in a microwell and incubated with 0.04 ml of 0.6 mM NAD⁺, L-phenylanine dehydrogenase (1 U), and 0.03 ml of 10 mM PMS . After 30 min, the concentration of Phe was determined on the basis of the absorbance at 663 nm assuming an extinction coefficient of 16,000 M⁻¹ cm⁻¹ for PMS-PPA complex. Absorbance values of the test solutions at 663 were measured by a microwell plate reader set at 660 nm. Blank values, obtained by following the same procedure as above but in the absence of L-phenylalanine dehydrogenase, were subtracted from each absorbance value. The net absorbance values (Test—BK) are strictly proportional to the amount of blood Phe in the range from 1 mg/dl (0.05 mM) to 15 mg/dl (1 mM). The detection limit is 0.06 mM. Besides the above described “end-point” procedure, the use of a kinetic analysis which limits the time for the test, is also possible. A particular advantage inherent the present calorimetric test is that known amounts of Phe can be added to the test solution at the end of the detection of endogeneous Phe. This procedure allows to check the efficiency of the enzyme used in each single test, the absence or presence of possible inhibitors or other interfering factors that may cause a “false negative” diagnosis.

### EXAMPLE 10

### Test for Neonatal Screening of Phenylketonuria on Solid Support

Preparation of the diagnostic stick: cellulose discs (2 cm diameter) were incubated in a 10 mM PMS solution dissolved in absolute ethanol and 0.4 mM NAD⁺. After 2 min the discs were dried in the dark under air stream (30° C.).

The blood sample was treated as reported in Example 9 and extracted with 0.5 ml of 1 M phosphate buffer, pH 8.5 (or 1 M Tris-HCl buffer, pH 8.5) and shaken for 10 min. After this step, 0.1 unit of L-phenylalanine dehydrogenase were added to 0.03 ml of the blood extract and loaded on the cellulose stick containing PMS and NAD⁺. After 60 min the amount of PMS-PPA which is stoichiometric to the blood Phe has been measured on the basis of the absorbance at 663 nm calculated by means of a reflectance apparatus set at 663 nm or by comparison of color scale. Linearity range and the detection limit are similar to those found in Example 9.

1. Method for quantitative and semi-quantitative determination of endogenous amino acids L-phenyalanine, L-tyrosine, L-3,4-dihydroxyphenylalanine and their corresponding keto-acids, phenylpiruvic acid, 3-hydroxyphenylpyruvic acid and 3,4-dihydroxyphenylpyruvic in biological fluids useful for diagnosis and monitoring of metabolic disorders of said amino acids or diseases involving said amino acids, comprising the following steps:
a) reaction of phenylpiruvic acid, 3-hydroxyphenylpyruvic acid and 3,4-dihydroxyphenylpyruvic, present as such in biological fluids or coming from the parent endogenous amino acids L-phenyalanine, L-tyrosine, L-3,4-dihydroxyphenylalanine by deamination, with an organic salt of phenazine derivatives in the presence of at least one alkaline buffer to give colored charge transfer complexes;
b) measurement of the absorbance values due to said charge transfer complexes in the wavelength range from 650 to 690 nm and quantification of the keto acids or amino acids concentrations in biological fluids.

2. Method according to claim 1, wherein the organic salt of phenazine derivatives is selected from the group consisting of phenazine methosulphate (PMS) or phenazine ethosulphate (PES).

3. Method according to claim 1, wherein the biological fluids are serum, blood or urine.

4. Method according to claim 1, wherein the biological fluid is serum and the endogeneous amino acids are previously deaminated by means of a chemical or enzymatic reaction.

5. Method according to claim 4, wherein the deamination reaction of the endogenous amino acids is catalyzed by the enzyme L-amino acid oxidase in the presence of an alkaline buffer.

6. Method according to claim 4, wherein, when the endogenous amino acid to be determined is L-phenyalanine, the deamination reaction is performed using the enzyme L-phenyalanine dehydrogenase in alkaline conditions and in the presence of a redox cofactor.

7. Method according to claim 6, wherein the redox cofactor is NAD⁺.

8. Method according to claim 1, wherein said at least one alkaline buffer is selected from the group consisting of potassium phosphate buffer, sodium phosphate buffer, TRIS-HCl buffer.

9. Method according to claim 1, wherein step a) is performed in solution or on solid support.

10. Method according to claim 1, wherein said metabolic disorders or diseases involving L-phenyalanine, L-tyrosine, L-3,4-dihydroxyphenylalanine are Phenylketonuria, Hyperphenylalaninaemia, Tyrosinaemia, Neuroblastoma, Parkinson disease.

11. Kit for diagnosis and monitoring of metabolic disorders of L-phenyalanine, L-tyrosine, L-3,4-dihydroxyphenylalanine or diseases involving said amino acids, based on quantitative detection of the parent keto-acids phenylpiruvic acid, 3-hydroxyphenylpyruvic acid and 3,4-dihydroxyphenylpyruvic comprising:
a) an organic salt of phenazine derivative in solution or adsorbed on solid support;
b) an alkaline buffer such as the extinction coefficients at 663 nm of complexes of phenylpiruvic acid, 3-hydroxyphenylpyruvic acid and 3,4-dihydroxyphenylpyruvic with the organic salt of phenazine derivatives are higher than 13,000 M⁻¹ cm⁻¹.

12. Kit according to claim 11, wherein the organic salt of phenazine derivative is selected from the group consisting of phenazine methosulphate (PMS) or phenazine ethosulphate (PES).

13. Kit according to claim 11, wherein the solid support is cellulose or equivalent absorbing materials.

14. Kit according to claim 11, wherein the alkaline buffer b) is potassium phosphate buffer or sodium phosphate buffer.

15. Kit according to claim 12, which also comprises:
c) a second alkaline buffer which lowers the extinction coefficients at 663 nm of complexes of phenylpiruvic acid, 3-hydroxyphenylpyruvic acid and 3,4-dihydroxyphenylpyruvic with an organic salt of phenazine derivatives below 3,600 M⁻¹ cm⁻¹.

16. Kit according to claim 15, wherein said second alkaline buffer c) is selected from the group consisting of TRIS-HCl buffer, glycine-NaOH buffer, borate buffer.

17. Kit according to anyone of the claims from 11 to 16, which also comprises:
d) a deaminating enzyme for L-phenyalanine, L-tyrosine, L-3,4-dihydroxyphenylalanine.

18. Kit according to claim 17, wherein the enzyme is L-amino acid oxidase.

19. Kit according to claim 17, wherein the enzyme is L-phenylalanine dehydrogenase in the presence of a redox coenzyme when the target amino acid is only L-phenyalanine.

20. Kit according to claim 19, wherein the redox coenzyme is NAD⁺.
